(19) United States
(12) Patent Application Publication
(10) Pub. No.: US 2019/0365484 A1
(43) Pub. Date: Dec. 5, 2019

(54) CANNULA SECURING ASSEMBLY FOR A MINIMALLY INVASIVE SURGICAL SYSTEM

(71) Applicant: SS Innovations China Co., Ltd., Hangzhou (CN)

(72) Inventor: Salman Kapadia, Barapatthar (IN)

(73) Assignee: SS Innovations China Co., Ltd., Hangzhou (CN)

(21) Appl. No.: 16/128,010

(22) Filed: Sep. 11, 2018

(30) Foreign Application Priority Data
Jun. 5, 2018 (IN) 201811020960

(51) Int. Cl. A61B 34/30; A61B 17/34; A61B 34/00; A61B 90/00

(52) U.S. Cl. CPC A61B 34/30; A61B 17/3421; A61B 17/00234; A61B 34/70; A61B 90/37; A61B 34/25

(57) ABSTRACT

A cannula securing assembly (300) for selectively engaging and disengaging a cannula (302) in a surgical system (100) is disclosed herein. The cannula securing assembly comprises of a housing (502) that includes at least one aperture (602) to receive a portion (410) of the cannula (302) and a locking plate (504) including at least one aperture (516) configured to engage with the portion (410) of cannula (302) received in the at least one aperture (602) of the housing (502). The locking plate (504) further includes a releasing means (512) at one end of the locking plate (504) wherein the releasing means (512) configured to be exposed on an exterior surface of the housing (502). The cannula securing assembly (300) further comprises of a compression means (508) affixed to a slot (520) on an interior surface of the housing (502) and configured to engage with the locking plate (504) where the compression means (508) is configured for biasing the locking plate (504) to facilitate engaging of the cannula (302) with the cannula securing assembly (300) and the releasing means (512) is configured for disconnecting the biasing of locking plate (504) to facilitate disengaging of the cannula (302) with the cannula securing assembly (300).

### FIELD OF THE INVENTION

The present invention generally relates to a minimally invasive surgical system. More particularly, the invention relates to a cannula securing assembly for selectively engaging and disengaging a cannula in the minimally invasive surgical system.

### BACKGROUND OF THE INVENTION

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as admissions of prior art.

Robotically assisted surgical systems have been adopted worldwide to replace conventional surgical procedures to reduce amount of extraneous tissue(s) that may be damaged during surgical or diagnostic procedures, thereby reducing patient recovery time, patient discomfort, prolonged hospital tenure, and particularly deleterious side effects. In robotically assisted surgeries, the surgeon typically operates a master controller at a surgeon console to seamlessly capture and transfer complex actions performed by the surgeon giving the perception that the surgeon is directly articulating surgical tools to perform the surgery. The surgeon operating on the surgeon console may be located at a distance from a surgical site or may be located within an operating theatre where the patient is being operated.

The robotically assisted surgeries have revolutionized the medical field and one of the fastest growing sector in medical device industry. However, the major challenge in robotically assisted surgeries is to ensure the safety and precision during the surgery. One of the key areas of robotically assisted surgeries is the development of surgical robots for minimally invasive surgery. Over the last couple of decades, surgical robots have evolved exponentially and has been a major area of innovation in the medical device industry.

The robotically assisted surgical systems comprises of multiple robotic arms aiding in conducting robotic surgeries. In most robotically assisted surgeries, one or more small incision is made in patient's body to provide entry point for various surgical instruments and endoscopic devices. To guide the various surgical instruments and endoscopic devices inside the patent's body during the robotically assisted surgery, first a cannula is inserted through the small incision point. The cannula provides access to the body cavity to perform various surgical procedures. Typically, cannula is elongated tube-like structure having one end with pointed structure which is inside the patient body during the surgical procedures. The other end of the cannula is attached on to the robotic arm assembly.

Performing surgeries with surgical instruments inside the patient body cavity through the cannula creates new challenges. One challenge is the stability of the cannula during the manipulation of surgical instruments during the surgery. Another challenge is the ease of locking and unlocking the cannula from the surgical system.

Additionally, in surgical procedures, the cannula is docked to an assembly on the robotic arm assembly. Such assembly has been convenient and effective for surgical procedures, but still require further improvements upon effective cannula mounting, locking/unlocking of cannula, stability of the cannula during a surgical operation, etc.

In the light of aforementioned challenges, there is a need for a robotic surgical system with a cannula securing assembly for selectively engaging and disengaging a cannula.

### SUMMARY OF THE INVENTION

A cannula securing assembly for selectively engaging and disengaging a cannula in a surgical system is disclosed herein. The cannula securing assembly comprises of a housing including at least one aperture to receive a portion of the cannula and a locking plate including at least one aperture configured to engage with the portion of cannula received in the at least one aperture of the housing. The locking plate further includes a releasing means at one end of the locking plate wherein the releasing means configured to be exposed on an exterior surface of the housing. The cannula securing assembly further comprises of a compression means affixed to a slot on an interior surface of the housing and configured to engage with the locking plate, wherein the compression means configured for biasing the locking plate to facilitate engaging of the cannula with the cannula securing assembly and the releasing means configured for disconnecting the biasing of locking plate to facilitate disengaging of the cannula with the cannula securing assembly.

A surgical system is also disclosed herein. The surgical system comprises of a cannula and a cannula securing assembly. The cannula securing assembly comprises of a housing including at least one aperture to receive a portion of the cannula and a locking plate including at least one aperture configured to engage with the portion of cannula received in the at least one aperture of the housing. The locking plate further includes a releasing means at one end of the locking plate wherein the releasing means configured to be exposed on an exterior surface of the housing. The cannula securing assembly further comprises of a compression means affixed to a slot on an interior surface of the housing and configured to engage with the locking plate, wherein the compression means configured for biasing the locking plate to facilitate engaging of the cannula with the cannula securing assembly and the releasing means configured for disconnecting the biasing of locking plate to facilitate disengaging of the cannula with the cannula securing assembly.

### BRIEF DESCRIPTION OF THE DRAWINGS

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings in which:

FIG. 1(a) is a schematic diagram of an operating room, illustrating multiple robotic arms of a robotic surgical system in accordance with an embodiment of the invention;

FIG. 1(b) illustrates a schematic diagram of a surgeon console of the robotic surgical system in accordance with an embodiment of the invention;

FIG. 1(c) illustrates a schematic diagram of a vision cart of the robotic surgical system in accordance with an embodiment of the invention;

FIG. 2(a) illustrates a perspective view of a tool interface assembly in accordance with an embodiment of the invention;

FIG. 2(b) illustrates an exploded view of the tool interface assembly in accordance with an embodiment of the invention;

FIG. 3(a) illustrates a perspective view of a cannula securing assembly attached to a cannula and a cannula sterile adaptor in accordance with an embodiment of the invention;

FIG. 3(b) illustrates an exploded view of the cannula securing assembly, cannula and cannula sterile adaptor in accordance with an embodiment of the invention;

FIG. 4 illustrates an exploded view of the cannula, the cannula sterile adaptor, and the cannula securing assembly in accordance with an embodiment of the invention;

FIG. 5(a) illustrates a side view of components of the cannula securing assembly in accordance with an embodiment of the invention;

FIG. 5(b) illustrates a top view of components of the cannula securing assembly in accordance with an embodiment of the invention; and

FIG. 6 illustrates a bottom view of a housing of the cannula securing assembly in accordance with an embodiment of the invention.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present invention. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this invention belongs. The apparatus, system, and examples provided herein are illustrative only and not intended to be limiting. The terms “a” and “an” herein do not denote a limitation of quantity, but rather denote the presence of at least one of the referenced item. Further, the term sterile barrier and sterile adapter denotes the same meaning and may be used interchangeably throughout the description. Embodiments of the invention will be described below in detail with reference to the accompanying drawings.

The invention relates to a robotic surgical system for minimally invasive surgery. The robotic surgical system will generally involve the use of multiple robotic arms. One or more of the robotic arms will often support a surgical tool which may be articulated (such as jaws, scissors, graspers, needle holders, micro dissectors, staple appliers, tackers, suction/irrigation tools, clip appliers, or the like) or non-articulated (such as cutting blades, cautery probes, irrigators, catheters, suction orifices, or the like). One or more of the robotic arms will often be used to support one or more surgical image/video capture devices such as an endoscope (which may be any of the variety of structures such as a laparoscope, an arthroscope, a hysteroscope, or the like), or optionally, some other imaging modality (such as ultrasound, fluoroscopy, magnetic resonance imaging, or the like).

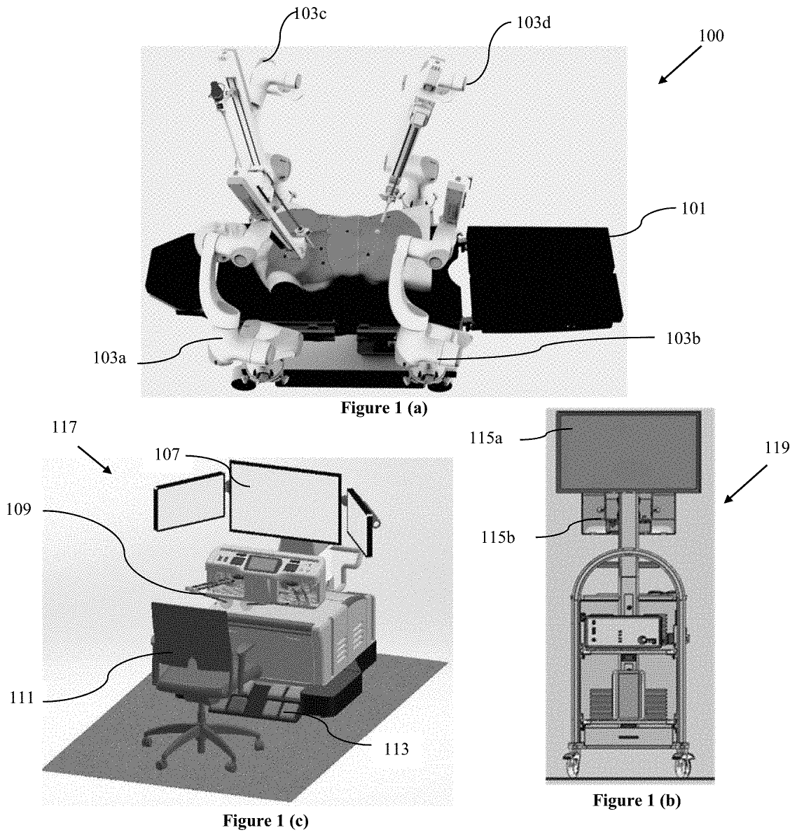

FIG. 1(a) illustrates a schematic diagram of multiple robotic arms of a robotic surgical system in accordance with an embodiment of the invention. Specifically, FIG. 1 illustrates the robotic surgical system (100) may have four robotic arms (103a), (103b), (103c), (103d) mounted around a patient cart (101). The four-robotic arms (103a), (103b), (103c), (103d) as depicted in FIG. 1 is for illustration purpose and the number of robotics arms may vary depending upon the type of surgery or the robotic surgical system. The four robotic arms (103a), (103b), (103c), (103d) may be mounted along the patient cart (101) and may be arranged in different manner but not limited to the robotic arms (103a), (103b), (103c), (103d) mounted on the patient cart (101) or the robotic arms (103a), (103b), (103c), (103d) separately mounted on a movable means or the robotic arms (103a), (103b), (103c), (103d) mechanically and/or operationally connected with each other or the robotic arms (103a), (103b), (103c), (103d) connected to a central body such that the robotic arms (103a), (103b), (103c), (103d) branch out of the central body (now shown).

FIG. 1(b) illustrates a schematic diagram of a surgeon console of the robotic surgical system in accordance with an embodiment of the invention. The surgeon console (117) may aid the surgeon to remotely operate the patient lying on the patient cart (101) by controlling the robotic arms (103a), (103b), (103c), (103d) around the body of the patient. The surgeon console (117) may be configured to control the movement of surgical instruments (not shown) while the instruments are inside the patient body. The surgeon console (117) may comprise of at least an adjustable viewing means (107) but not limited to 2D/3D monitors, wearable viewing means (not shown) and in combination thereof. The surgeon console (117) may be equipped with multiple displays which would not only show 3D high definition (HD) endoscopic view of a surgical site at the patient cart (101) but may also shows additional information from various medical equipment's which surgeon may use during the robotic surgery. Further, the viewing means (107) may provide various modes of the robotic surgical system (100) but not limited to identification and number of robotic arms attached, current surgical instrument type attached, current instrument tool tip position, collision information along with medical data like ECG, ultrasound display, fluoroscopic images, CT, MRI information. The surgeon console (117) may further comprise of mechanism for controlling the robotics arms but not limited to one or more hand controllers (109), one or more-foot controllers (113), a clutch mechanism (not shown), and in combination thereof The hand controllers (109) at the surgeon console (117) are required to seamlessly capture and transfer complex actions performed by surgeon giving the perception that the surgeon is directly articulating the surgical tools. The different controllers may require for different purpose during the surgery. In some embodiments, the hand controllers (109) may be one or more manually-operated input devices, such as a joystick, exoskeletal glove, a powered and gravity-compensated manipulator, or the like. These hand controllers (109) control teleoperated motors which, in turn, control the movement of the surgical instruments attached to the robotic arms. The surgeon may sit on a resting apparatus such as a chair (111), as depicted in FIG. 1(b), while controlling the surgeon console (117). The chair (111) may be adjustable with means in height, elbow rest, and the like according to the ease of the surgeon and also various control means may be provided on the chair (111). Further, the surgeon console (117) may be at a single location inside an operation theatre or may be distributed at any other location in the hospital provided connectivity to the robotics arms is maintained.

FIG. 1(c) illustrates a schematic diagram of a vision cart of the robotic surgical system in accordance with an embodiment of the invention. The vision cart (119) may be configured to display the 2D and/or 3D view of the operation captured by an endoscope. The vision cart (119) may be adjusted at various angles and heights depending upon the ease of view. The vison cart (119) may have various functionality but not limited to providing touch screen display, preview/recording/playback provisions, various inputs/outputs means, 2D to 3D converters, and the like. The vision cart (119) may include a vision system or displays (115a), (115b) that enables a spectator or other non-operating surgeons/assistants to view a surgical site from outside the patient's body. One of the robotics arms typically engage a camera that has a video-image-capture function (i.e., an endoscopic camera) for displaying the captured images on the vision cart (119). In some robotic surgical system configurations, the camera includes optics that transfer the images from the distal end of the camera to one or more imaging sensors (e.g., CCD or CMOS sensors) outside of the patient's body. Alternatively, the imaging sensor(s) may be positioned at the distal end of the camera, and the signals produced by the sensor(s) may be transmitted along a wire or wirelessly for processing and display on the vision cart (119).

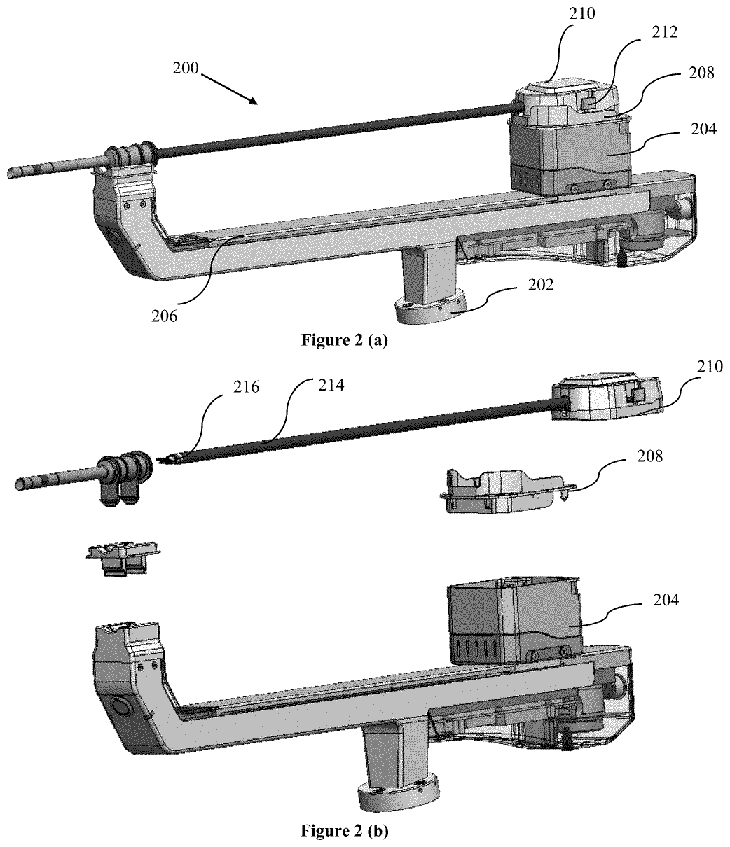

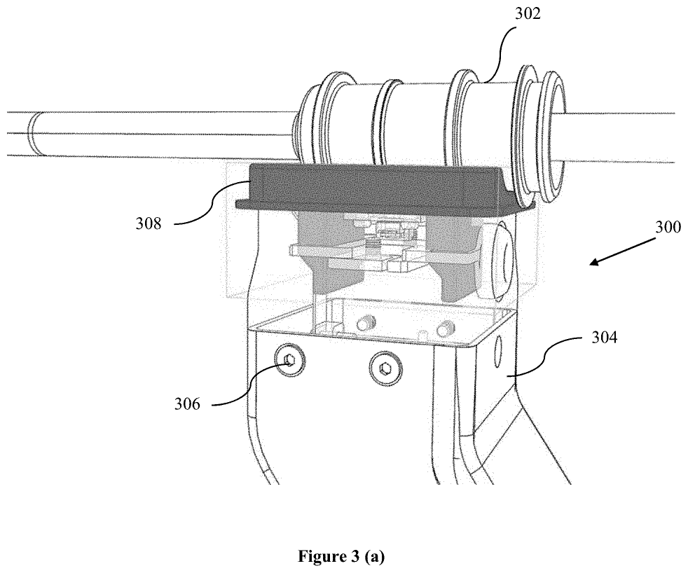

FIG. 2(a) illustrates a perspective view of the tool interface assembly and FIG. 2(b) illustrates an exploded view of the tool interface assembly in accordance with an embodiment of the invention. The tool interface assembly (200) may be mounted on any of the robotic arm of the robotic surgical system (100). The tool interface assembly (200) may be the main component for performing the robotic surgery on a patient.

The tool interface assembly (200), as depicted by any of the FIG. 2(a) or FIG. 2(b), may comprise of an Arm and Tool Interface (ATI) connector (202) which facilitates the tool interface assembly (200) to operationally connect with the robotic arm (shown in FIG. 1(a)). The tool interface assembly (200) may further comprise of an actuator assembly (204) mounted on a guiding mechanism and capable of linearly moving along the guiding mechanism. The guiding mechanism depicted in FIGS. 2(a) and 2(b) is a guide rail (206). The movement of the actuator assembly (204) along the guide rail (206) is controlled by the surgeon with the help of hand controllers on the surgeon console (117) as shown in FIG. 1(b). A sterile adapter assembly (208) is releasably mounted on the actuator assembly (204) to separate a non-sterile part of the robotic arm from a sterile surgical tool assembly (210). A locking mechanism (not shown) may be provided to releasably lock and unlock the sterile adapter assembly (208) with the actuator assembly (204). The sterile adaptor assembly (208) may detachably engage with the actuator assembly (204) which drives and controls the sterile surgical instrument in a sterile field. In another embodiment, the sterile surgical tool assembly (210) also may be releasably lock/unlock or engages/disengages with the sterile adapter assembly (208) by means of a push button (212).

The sterile surgical tool assembly (210) may include a shaft (214) and end effectors (216). The end effector (216) may comprises of a surgical instrument integrated with the end effector (216) or the end effector (216) may be configured to include an attachment means to attach a surgical instrument (not shown) where the end effector (216) may act as a pluggable device. The surgical instrument may be associated with one or more surgical tasks, such as a forcep, a needle driver, a shears, a bipolar cauterizer, a tissue stabilizer or retractor, a clip applier, an anastomosis device, an imaging device (e.g., an endoscope or ultrasound probe), and the like. Some surgical instruments further provide an articulated support (sometimes referred to as a “wrist”) for the sterile surgical tool assembly (210) such that the position and orientation of the surgical tool assembly (210) may be manipulated with one or more mechanical degrees of freedom in relation to the instrument's shaft (214). Further, the end effectors (216) may include a functional mechanical degree of freedom, such as jaws that open or close, or a knife that translates along a path. The sterile surgical tool assembly (210) may also contain stored (e.g., on a memory inside the instrument) information that may be permanent or may be updatable by the robotic surgical system (100).

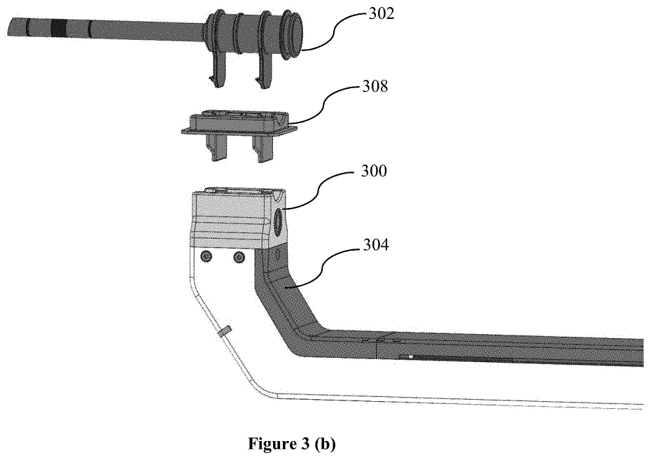

FIG. 3(a) illustrates a perspective view of a cannula securing assembly attached to a cannula and a cannula sterile adaptor in accordance with an embodiment of the invention. The cannula securing assembly (300) may be affixed to the body of the tool interface assembly (200) and may be configured to grip or secure the cannula (302) such that cannula (302) is stable while performing surgical operations. The cannula securing assembly (300) may be affixed to a mount (304) of the tool interface assembly (200) by bolts (306). Alternatively, the cannula securing assembly (300) may be riveted, screwed or a combination thereof to the mount (304) of the tool interface assembly (200). According to a specific embodiment of the invention, opposite sides of the cannula securing assembly (300) may be bolted on respective opposite sides of the mount (304) of the tool interface assembly (200).

The cannula securing assembly (300) may be made of any suitable resilient material such as a metal or an alloy. The material for the cannula securing assembly (300) can be selected from a group consisting aluminium, steel, iron, nickel, copper, zinc, tin, or any combination thereof In accordance to a specific embodiment of the invention, the cannula securing assembly (300) is made of aluminium. The cannula securing assembly (300) may be painted or may have a protective coating such as alloy coating. In accordance with an embodiment, the process of anodizing may be used to coat the cannula securing assembly (300) such as to form a protective coating of aluminum oxide on the surface of the cannula securing assembly (300). The cannula securing assembly (300) may be of any suitable size that can be conveniently attached to the mount (304) of the tool interface assembly (200) without affecting ease of the surgical operation. The cannula securing assembly (300) may be of a suitable thickness providing sufficient strength.

The cannula securing assembly (300) may be of any suitable shape (suitably similar to the mount (304) of the tool interface assembly (200)) such that the ease of affixing the cannula securing assembly (300) is maintained. In accordance with an embodiment of the invention, the cannula securing assembly (300) is substantially of a square shape where a bottom end of the cannula securing assembly (300) is substantially larger than a top end of the cannula securing assembly (300) and the body of the cannula securing assembly (300) is substantially tapered towards the top end of the cannula securing assembly (300). Detailed explanation of the cannula securing assembly (300) is provided in description of accompanying figures.

Referring now about the cannula, the cannula (302) may comprise of a hollow body. The cannula (302) is fixed to the shaft (214) (shown in FIG. 2(b)) at desired angle and precludes shifting, twisting or any axial movement of the shaft (214) once received by the cannula (302). The cannula (302) may be inserted through an opening in a patient's body to a surgical site. For example, a first end of cannula (explained later) may be inserted through an opening, such as, for example, an incision, natural orifice, or port, to a surgical site. A surgical instrument in the exemplary embodiment of FIG. 2(b), may be inserted through the cannula (302) to the surgical site. For example, an instrument may be inserted into a second end of cannula and advanced through the hollow body and the first end of the cannula (302) to the surgical site.

The cannula (302) may be made of any suitable resilient material such as a metal or an alloy. The material for the cannula (302) can be selected from a group consisting aluminium, steel, iron, nickel, copper, zinc, tin, or any combination thereof. In accordance to a specific embodiment of the invention, the cannula (302) is made of aluminium. The cannula (302) may be painted or may have a protective coating such as alloy coating. In accordance with an embodiment, the process of anodizing may be used to coat the cannula (302) such as to form a protective coating of aluminum oxide on the surface of the cannula (302). The cannula (302) may be of any suitable size that can be conveniently attached to the cannula securing assembly (300) without affecting ease of the surgical operation. The cannula (302) may be of a suitable thickness providing sufficient strength.

The cannula (302) may be of any suitable shape such that the ease of affixing the cannula (302) is maintained. In accordance with an embodiment of the invention, the cannula (302) is a long elongated hollow tube attached to a bowl section. Detailed explanation of the cannula (302) is provided in description of accompanying figures.

A cannula sterile adaptor (308) may be mounted between the cannula securing assembly (300) and the cannula (302), with the cannula sterile adaptor (308) being connected or sealed (may be thermal sealing) to a drape (not shown) such the cannula sterile adaptor (302) and the drape may form a boundary between a sterile region and non-sterile region. For better understanding, the part of cannula sterile adaptor (308) that is connected or sealed to the surgical drape (not shown) with a sterile region on the side of the drape facing cannula (302) and the non-sterile region on the side of the drape facing cannula securing assembly (300).

The cannula sterile adaptor (308) may be made of a relatively rigid material that provides structural support for the cannula sterile adaptor (308) when the cannula sterile adaptor (308) is mounted to the cannula securing assembly (300), as well as for the cannula (300) when the cannula (300) is mounted to the cannula securing assembly (300). Further, the relatively rigid material can be a smooth, low friction material, which may facilitate alignment and insertion of cannula (302) into cannula sterile adaptor (308) by providing a low friction surface over which attachment portions (explained later) may easily slide. In addition, the cannula sterile adaptor (308) may be configured to accommodate forces applied between cannula (302) and the cannula securing assembly (300), such as clamping forces and body wall forces, when cannula (303) is mounted to the cannula securing assembly (300).

The cannula sterile adaptor (308) may be made of a plastic material, such as, for example, polycarbonate, acrylonitrile butadiene styrene (ABS), polycarbonate/ABS, polyurethane, and other plastics familiar to one of ordinary skill in the art. The low friction surface may also assist with latching the cannula (302) to the cannula securing assembly (300) by facilitating sliding of cannula (302) when a latching force supplied by the cannula securing assembly (300) draws the cannula (302) into a mounting position during latching. According to a specific embodiment, the cannula sterile adaptor (308) may be treated with a lubricant to facilitate insertion and/or removal of the cannula (302). The lubricant may be, for example, a dry coating of polytetrafluoroethylene (PTFE) or other lubricant familiar to one of ordinary skill in the art that is applied to a surface of cannula sterile adaptor (308). According to a specific embodiment, the cannula sterile adaptor (308) is a hat like structure which encloses or sits over the top end of the cannula securing assembly (300). FIG. 3(b) illustrates an exploded view of the cannula securing assembly (300), the cannula (302) and the cannula sterile adaptor (308) in distributed manner. Detailed explanation of the assembly is provided in description of accompanying figures.

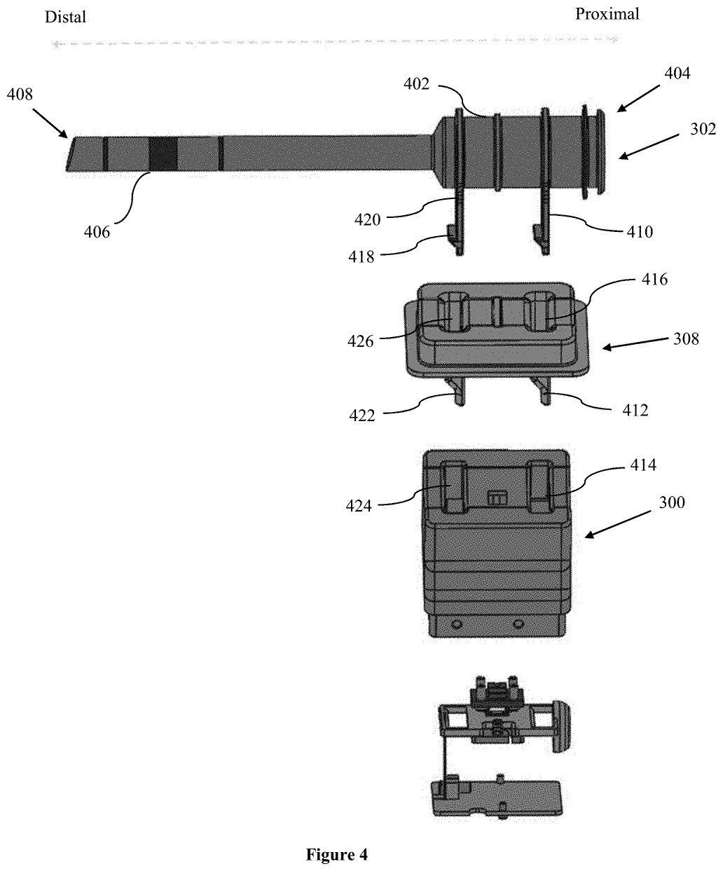

FIG. 4 illustrates an exploded view of the cannula, the cannula sterile adaptor, and the cannula securing assembly in accordance with an embodiment of the invention. The cannula (302) may comprise of a hollow body. The cannula (302) is fixed to the shaft (214) (shown in FIG. 2(b)) at desired angle and precludes shifting, twisting or any axial movement of the shaft (214) once received by the cannula (302). The cannula (302) may be inserted through an opening in a patient's body to a surgical site.

The cannula (302) may include a bowl section (402) forming a proximal end (404) of cannula (302), and a tube (406) may extend from the bowl section (402) to a distal end (408) of cannula (302). The proximal and distal directions with respect to the orientation of FIG. 4 are labeled. According to a specific embodiment as illustrated in FIG. 4, the tube (406) may have a length L, the distal end (408) and proximal end (404) may have a diameter D, Dᵢ, and the bowl section (402) having a circumference C, each of which may vary depending on a desired application of the cannula (302). Further, as shown in the specific embodiment of FIG. 4, the tube (406) may be straight, although the exemplary cannula embodiments described herein are not limited to a straight tube. For example, the tube (406) may instead be a curved tube having a substantially curved longitudinal axis. The cannula (302) may also comprise of an attachment portion (410) which encloses the bowl section (402) and may have a protruding end. The protruding end of the attachment portion (410) may be provided with locking prongs (418) which helps the attachment portion (410) in engaging or disengaging from the cannula securing assembly (300). According to a specific embodiment, a pair of attachment portions (410), (420) are illustrated in FIG. 4.

The cannula sterile adaptor (308) may be connected to the cannula securing assembly (300), such as by inserting holding portions (412), (422) of the cannula sterile adaptor (308) into respective apertures (414), (424) of the cannula securing assembly (300). According to a specific embodiment as illustrated in FIG. 4, a pair of holding portion (412), (422) and a pair of respective recesses (414), (424) are provided. The invention may function with one holding portion and one recess also. According to an embodiment, the shape of the holding portion (412), (422) corresponds to the shape of the recess (414), (424) of the cannula securing assembly (300) such as to facilitate mounting of the cannula sterile adaptor (308) in the cannula securing assembly (300).

The cannula sterile adaptor (308) may facilitate forming a boundary between a sterile region and non-sterile region, which is discussed earlier. For example, a surgical drape (not shown) may be attached to the cannula sterile adaptor (308) to separate a sterile side from a non-sterile side of the drape.

The attachment portions (410), (420) of the cannula (302) may be structured to fit inside openings (416), (426) of the cannula sterile adaptor (308) so that the cannula (302) remains on sterile side of the drape. Further, when the cannula sterile adaptor (308) has been connected to the cannula securing assembly (300), and attachment portions (410), (420) are inserted into the openings (416), (426) of the cannula sterile adaptor (308), the cannula (302) may also be connected to the cannula securing assembly (300) so that the cannula (302) may be held by the cannula securing assembly (300) during a surgical procedure. The invention may function with one attachment portion and one opening also.

Alternatively, the attachment portion (410), (420) of the cannula (302) may be structured to fit inside the recess (414), (424) of the cannula securing assembly (300) in case the cannula sterile adaptor (308) is not used in surgical operations.

According to an embodiment, the shape of the attachment portions (410), (420) corresponds to the shape of the openings (416), (426) of the cannula sterile adaptor (308) such as to facilitate mounting of the cannula (302) in the cannula sterile adaptor (308).

According to an exemplary embodiment, the attachment portions (410), (420) may include locking prongs (418), such as on one side of attachment portion (410), (420) (as shown in FIG. 4), to assist with mounting the cannula (302) to the cannula securing assembly (300). Locking prongs (418) may be configured to facilitate mounting of the cannula (302) to the cannula sterile adaptor (308) and thereon to the cannula securing assembly (300). For example, an angle of locking prongs (418) may be selected to facilitate an application of a large amount of force between the attachment portions (410), (420), cannula sterile adaptor (308), and cannula securing assembly (300) when cannula (302) is mounted, but also facilitate release of the attachment portions (410), (420). According to a specific embodiment, the locking prongs (418) may have a protruding profile substantially orthogonal to the attachment portions (410), (420) as illustrated in FIG. 4.

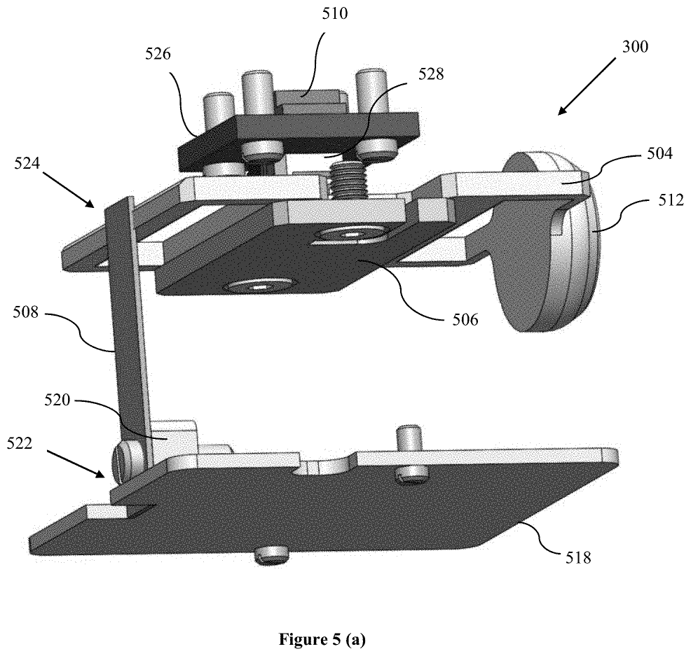

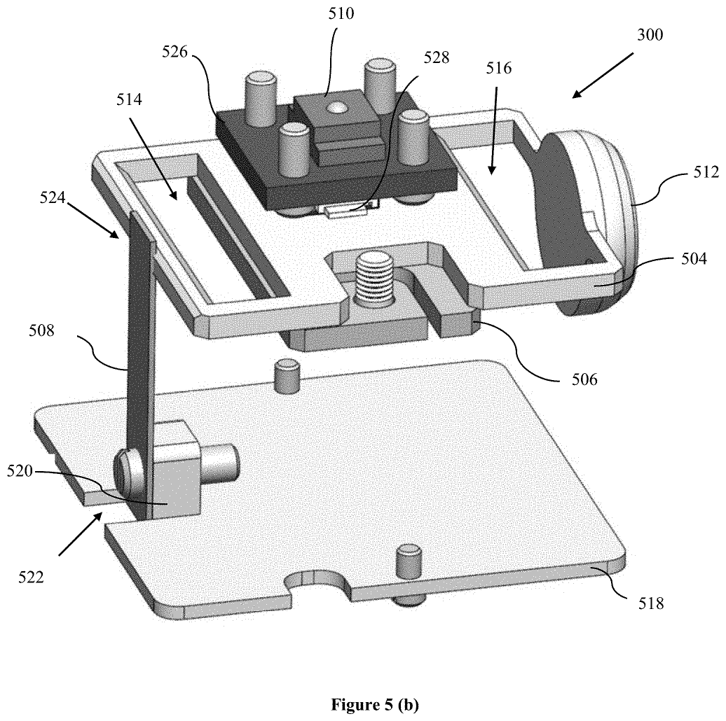

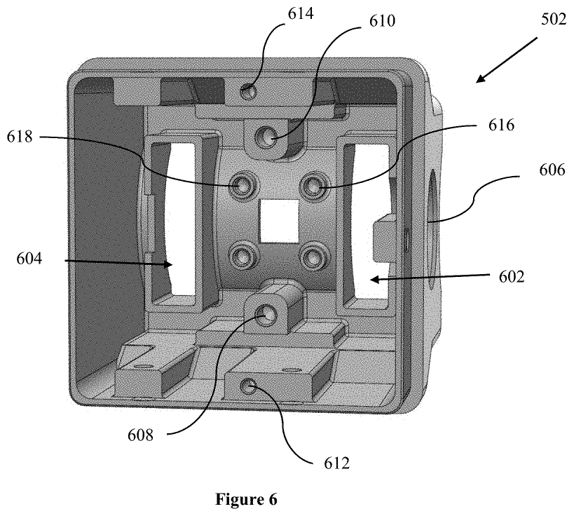

FIGS. 5(a), 5(b), and 6 illustrates components of the cannula securing assembly in accordance with an embodiment of the invention. The cannula securing housing (300) may comprise of a housing (502) (shown in FIG. 6), a locking plate (504), a retainer plate (506), a compression means (508), and one or more sensors (510).

Referring now to FIG. 6, the housing (502) may be a shell for enclosing the parts of the cannula securing assembly (300). The housing also includes an aperture (602) (shown in FIG. 6) (such as apertures (414, 424)) to receive the holding portion (412) of the cannula sterile adaptor (308) having the attachment portion (410) of the cannula (302) contained therein. According to a specific embodiment as illustrated in FIG. 6, a pair of aperture (602), (604) is provided to receive a pair of holding portions (412), (422). The invention may function with one recess and one holding portion also. According to an embodiment, the shape of the aperture (602), (604) corresponds to the shape of the holding portion (412), (422) of the cannula sterile adaptor (308) such as to facilitate mounting of the cannula (302) in the housing (502) of the cannula securing assembly (300).

The housing (502) may be made of any suitable resilient material such as a metal or an alloy. The material for the housing (502) can be selected from a group consisting aluminium, steel, iron, nickel, copper, zinc, tin, or any combination thereof. In accordance to a specific embodiment of the invention, the housing (502) is made of aluminium. The housing (502) may be painted or may have a protective coating such as alloy coating. In accordance with an embodiment, the process of anodizing may be used to coat the housing (502) such as to form a protective coating of aluminum oxide on the surface of the housing (502). The housing (502) may be of any suitable size that can be conveniently attached to the mount (304) of the tool interface assembly (200) without affecting ease of the surgical operation. The housing (502)) may be of a suitable thickness providing sufficient strength. The housing (502) may be of any suitable shape (suitably similar to the mount (304) of the tool interface assembly (200)) such that the ease of affixing the aforesaid is maintained. In accordance with an embodiment of the invention, the housing (502) is substantially of a square shape where a bottom end of the housing (502) is substantially larger than a top end of the housing (502) and the body of the housing (502) is substantially tapered towards the top end of the housing (502).

Referring now to FIGS. 5(a) and 5(b), the locking plate (504) may be a flat plate containing at least one aperture (514) configured to receive and engage with portions received by the aperture (602) of the housing (502). According to a specific embodiment as illustrated in FIGS. 5(a) and 5(b), a pair of aperture (514), (516) are provided in the locking plate (504).

The pair of apertures (514), (516) are configured to receive the holding portion (412), (422) of the cannula sterile adaptor (308) having the attachment portions (410), (420) of the cannula (302) contained therein. According to a specific embodiment as illustrated in FIGS. 5(a) and 5(b), a pair of aperture (602), (604) are provided to engage with the locking prongs (418) of the attachment portions (410), (420) of the cannula (302). The invention may function with one aperture and one holding portion also. According to an embodiment, the shape of the apertures (514), (516) corresponds to the shape of the holding portions (412), (422) of the cannula sterile adaptor (308) and the attachment portions (410), (420) of the cannula (302), such as to facilitate mounting of the cannula (302) in the housing (502) of the cannula securing assembly (300). According to an embodiment, the diameter of the apertures (514), (516) of the locking plate (504) corresponds with the diameter of the apertures (602), (604). The locking plate (504) is capable of translation motion within the housing (502).

The locking plate (504) may be made of any suitable resilient material such as a metal or an alloy. The material for the locking plate (504) can be selected from a group consisting aluminium, steel, iron, nickel, copper, zinc, tin, or any combination thereof. In accordance to a specific embodiment of the invention, the locking plate (504) is made of aluminium. The locking plate (504) may be painted or may have a protective coating such as alloy coating. In accordance with an embodiment, the process of anodizing may be used to coat the locking plate (504) such as to form a protective coating of aluminum oxide on the surface of the locking plate (504). The locking plate (504) may be of any suitable size that can be conveniently attached to the housing (502) without affecting ease of the surgical operation. The locking plate (504) may be of a suitable thickness providing sufficient strength.

The locking plate (504) also may comprise of a releasing means (512) at one end of the locking plate (504) where the releasing means is configured to be exposed on an exterior surface of the housing (502). According to an embodiment, the releasing means (512) is shaped like a button and when pressed or applied pressure can be configured to disengage the cannula (302) with the cannula securing assembly (300). According to a specific embodiment, the releasing means (512) is a shutter-release button.

Referring now to FIG. 6, the housing (502) comprises of an opening (606) for receiving the releasing means (512) of the locking plate (504). According to an embodiment, the opening (606) is circular in shape and the diameter of the opening (606) is substantially larger than the diameter of the releasing means (512) of the locking plate (504) such that the releasing means (512) fits well within the opening (606).

Referring back to FIGS. 5(a) and 5(b), the retainer plate (506) is affixed to an interior surface of the housing (502) such that the retainer plate (506) is fixed over the locking plate (504) for facilitating a reciprocating movement of the locking plate (504). The retainer plate (506) is configured to fix the locking plate (504) in one position such that the locking plate (504) is secured within the housing (502). The retainer plate (506) may be bolted to the housing (502). Alternatively, the retainer plate (506) may be riveted, screwed or a combination thereof to the housing (502). As illustrated in FIG. 6, slots (608), (610) are provided on an internal surface within the housing (502) for the retainer plate (506) to be bolted therein.

The retainer plate (506) may be made of any suitable resilient material such as a metal or an alloy. The material for the retainer plate (506) can be selected from a group consisting aluminium, steel, iron, nickel, copper, zinc, tin, or any combination thereof. In accordance to a specific embodiment of the invention, the retainer plate (506) is made of aluminium. The retainer plate (506) may be painted or may have a protective coating such as alloy coating. In accordance with an embodiment, the process of anodizing may be used to coat the retainer plate (506) such as to form a protective coating of aluminum oxide on the surface of the retainer plate (506)). The retainer plate (506) may be of any suitable size that can be conveniently attached to the housing (502) without affecting ease of the surgical operation. The retainer plate (506) may be of a suitable thickness providing sufficient strength.

The cannula securing housing (300) may comprise of a back cover (518) configured to close the housing from one end as illustrated in FIGS. 5(a) and 5(b). According to an embodiment, the back cover (518) may be a flat plate. The back cover (518) may be bolted to the housing (502). Alternatively, the back cover (518) may be riveted, screwed or a combination thereof to the housing (502). As illustrated in FIG. 6, slots (612), (614) are provided on an internal surface within the housing (502) for the back cover (518) to be bolted therein.

The back cover (518) may also comprise of a protruding slot (520) on an internal surface of the back cover (518) such that when the housing (502) is closed with the back cover (518), the protruding slot (520) remains within the housing (502).

The back cover (518) may be made of any suitable resilient material such as a metal or an alloy. The material for the back cover (518) can be selected from a group consisting aluminium, steel, iron, nickel, copper, zinc, tin, or any combination thereof. In accordance to a specific embodiment of the invention, the back cover (518) is made of aluminium. The back cover (518) may be painted or may have a protective coating such as alloy coating. The back cover (518) may be of any suitable size that can be conveniently attached to the housing (502) without affecting ease of the surgical operation. The back cover (518) may be of a suitable thickness providing sufficient strength.

The compression means (508) may be provided for biasing the locking plate (504) to facilitate engaging of the cannula (302) with the housing (502) of the cannula securing assembly (300). According to an embodiment, the compression means (508) is a flat spring.

The compression means (508) at a first end (522) is bolted to the protruding slot (520) of the back cover (518) such that the compression means (508) is fixed within the housing (502). Alternatively, the compression means (508) may be riveted, screwed or a combination thereof to the protruding slot (520) of the back cover (518). The compression means (508) at a second end (524) engages with the locking plate (504) such that the compression means (508) exerts a tension on the locking plate (504) wherein the locking plate (504) stays in a locked position. When the locking plate (504) is in the locked position, the cannula (302) is engaged within the pair of aperture (514), (516) of the locking plate (504).

Herein, biasing the locking plate (504) may be referred as the spring applying a biasing force on the locking plate (504) at all times for the locking plate (504) to engage the cannula (302) within the housing (502) of the cannula securing assembly (300). Further, the spring may apply the biasing force on the locking plate (504) such that the locking prongs (418) of the attachment portions (410), (420) of the cannula (302) are constantly engaged within the pair of aperture (514), (516) of the locking plate (504).

On the other hand, when the releasing means (512) is pressed, the tension applied by the compression means (508) is disengaged or interrupted which facilitates the disengagement of cannula (302) from the pair of aperture (514), (516) of the locking plate (504). Herein, the biasing force may be depressed or released such that the locking prongs (418) of the attachment portions (410), (420) of the cannula (302) may be disengaged from the pair of aperture (514), (516) of the locking plate (504) and the cannula (302) pops out as a result of the disengagement. This stage may be referred as when the locking plate (504) is in an unlocked position.

Further, one or more sensors (510) may be positioned towards a top end of the housing (502) and may be configured to measure force exerted on the cannula (302) and detect presence of the cannula (302) within the housing (502). According to a specific embodiment, force sensors may be used. The one or more sensors (510) may be positioned on a PCB (printed circuit board) (526) which may be bolted to an internal surface within the housing (502). Alternatively, the PCB (526) may be riveted, screwed or a combination thereof to the housing (502). As illustrated in FIG. 6, slots (616), (618) are provided on an internal surface within the housing (502) for the PCB (526) to be bolted therein.

The PCB (526) may be fixed to the locking plate (504) with the help of a connector (528). As illustrated by FIGS. 5(a) and 5(b), a force sensor is connected on the PCB (526) to measure the force applied on the cannula (302).

According to an exemplary embodiment, the one or more sensors (510) measures the force exerted by the surgeon on the cannula (302) and also on the tissues of the patient's body wall around the cannula (302) during a surgery. During the surgery, the force exerted by the surgeon on the cannula (302) is transferred to the one or more sensors (510) by means of a shaft (214) connected at a proximal end (404) of the cannula (302). The shaft (214) presses the one or more sensors (510) and the force is detected by the one or more sensors (510) and signal(s) may be conveyed to the display (115a) of the vison cart 119 (as shown in FIG. 1(c)). Further, the signal may also be displayed at the viewing means (107) at the surgeon console (as shown in FIG. 1(b)). Furthermore, a threshold may be set and whenever the force exerted on the cannula (302) increases above the threshold, an alert by way of an alarm or a message is displayed to the surgeon or assistants near the surgeon. By way of example, the sensor (510) may also be a load cell sensor, which measures the deformation of the tissues of the patient's body wall around the cannula (302) into an electrical signal and send an alert to the surgeon if the deformation is above a threshold.

According to a specific embodiment, the attachment portions (410), (420) are the portion of cannula (302) that is received by the housing (502).

A surgical system is also disclosed herein. The surgical system comprises of a cannula and a cannula securing assembly. The cannula securing assembly comprises of a housing including at least one aperture to receive a portion of the cannula and a locking plate including at least one aperture configured to engage with the portion of cannula received in the at least one aperture of the housing. The locking plate further includes a releasing means at one end of the locking plate wherein the releasing means configured to be exposed on an exterior surface of the housing. The cannula securing assembly further comprises of a compression means affixed to a slot on an interior surface of the housing and configured to engage with the locking plate, wherein the compression means configured for biasing the locking plate to facilitate engaging of the cannula with the cannula securing assembly and the releasing means configured for disconnecting the biasing of locking plate to facilitate disengaging of the cannula with the cannula securing assembly.

The foregoing descriptions of exemplary embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present invention.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

We claim:

1. A cannula securing assembly (300) for selectively engaging and disengaging a cannula (302) in a surgical system (100), the cannula securing assembly comprising:
a housing (502) including at least one aperture (602) to receive a portion (410) of the cannula (302);
a locking plate (504) including at least one aperture (516) configured to engage with the portion (410) of cannula (302) received in the at least one aperture (602) of the housing (502) and a releasing means (512) at one end of the locking plate (504), wherein the releasing means (512) configured to be exposed on an exterior surface of the housing (502); and
a compression means (508) affixed to a slot (520) on an interior surface of the housing (502) and configured to engage with the locking plate (504),
wherein the compression means (508) configured for biasing the locking plate (504) to facilitate engaging of the cannula (302) with the cannula securing assembly (300) and the releasing means (512) configured for disconnecting the biasing of locking plate (504) to facilitate disengaging of the cannula (302) with the cannula securing assembly (300).

2. The cannula securing assembly (300) as claimed in claim 1, further comprising one or more sensors (510) to measure force exerted on the cannula (302) and detect presence of the cannula (302) in the cannula securing assembly (300).

3. The cannula securing assembly (300) as claimed in claim 2, wherein the one or more sensors (510) positioned on a Printed Circuit Board (PCB) (526) wherein the PCB (526) is affixed to an internal surface of the housing (502).

4. The cannula securing assembly (300) as claimed in claim 1, further comprising a retainer plate (506) affixed to an interior surface of the housing (502) such that the retainer plate (506) is fixed over the locking plate (504) for facilitating a reciprocating movement of the locking plate (504).

5. The cannula securing assembly (300) as claimed in claim 1, wherein the compression means (508) is a flat spring.

6. The cannula securing assembly (300) as claimed in claim 1, wherein the cannula securing assembly (300) is made of aluminium.

7. The cannula securing assembly (300) as claimed in claim 1, wherein the cannula securing assembly (300) configured to receive a cannula sterile adaptor (308) such that the cannula sterile adaptor (308) is positioned between the cannula securing assembly (300) and cannula (302).

8. The cannula securing assembly (300) as claimed in claim 1, wherein the cannula sterile adaptor (308) is connected to a drape such that the cannula sterile adaptor (308) and the drape forms a boundary between a sterile region and non-sterile region.

9. A surgical system (100), comprising;
a cannula (302); and
a cannula securing assembly (300) comprising:
a housing (502) including at least one aperture (602) to receive a portion (410) of the cannula (302);
a locking plate (504) including at least one aperture (516) configured to engage with the portion (410) of cannula (302) received in the at least one aperture (602) of the housing (502) and a releasing means (512) at one end of the locking plate (504), wherein the releasing means (512) configured to be exposed on an exterior surface of the housing (502); and
a compression means (508) affixed to a slot (520) on an interior surface of the housing (502) and configured to engage with the locking plate (504),
wherein the compression means (508) configured for biasing the locking plate (504) to facilitate engaging of the cannula (302) with the cannula securing assembly (300) and the releasing means (512) configured for disconnecting the biasing of locking plate (504) to facilitate disengaging of the cannula (302) with the cannula securing assembly (300).

10. The surgical system (100) as claimed in claim 9, further comprising a cannula sterile adaptor (308) configured to be disposed between the cannula (302) and cannula securing assembly (300), wherein the cannula sterile adaptor (308) is connected to a drape such that the cannula sterile adaptor (308) and the drape forms a boundary between a sterile region and non-sterile region.

11. The surgical system (100) as claimed in claim 9, wherein the cannula (302) comprises a bowl section (402) forming a proximal end (404) of the cannula (302) and a tube (406) extending from the bowl section (402) to a distal end (408) of the cannula (302).

12. The surgical system (100) as claimed in claim 9, wherein the cannula (302) further comprises an attachment portion (410), (420) having locking prongs (418) configured to engage or disengage the cannula (302) from the cannula securing assembly (300).

13. The surgical system (100) as claimed in claim 9, wherein the cannula securing assembly (300) comprises a retainer plate (506) affixed to an interior surface of the housing (502) such that the retainer plate (506) is fixed over the locking plate (504) for facilitating a reciprocating movement of the locking plate (504).
